(19) United States
(12) Patent Application Publication
(10) Pub. No.: US 2018/0179192 A2
(43) Pub. Date: Jun. 28, 2018

(54) NOVEL COMPLEXES FOR THE SEPARATION OF CATIONS

(71) Applicants: UNIVERSITE PARIS-SUD, Orsay (FR); COMMISSARIAT A L'ENERGIE ATOMIQUE ET AUX ENERGIES ALTERNATIVES, Paris (FR); CENTRE NATIONAL DE LA RECHERCHE SCIENTIFIQUE, Paris (FR)

(72) Inventors: Vincent HUC, Orsay (FR); Pascal VIEL, Meudon (FR); Ekaterina SHILOVA, Marseille (FR)

(21) Appl. No.: 15/301,423

(22) PCT Filed: Apr. 3, 2015
(86) PCT No.: PCT/EP2015/097016

§ 371 Date: Oct. 3, 2016

(30) Foreign Application Priority Data
Apr. 3, 2014 (FR) 1452958

(51) Int. Cl. C07D 405/14; B01J 20/26; B01J 20/30; C07F 9/40; C08L 39/08; C08L 33/02; C02F 1/28; C02F 1/56

(52) U.S. Cl. CPC C07D 405/14; B01J 20/267; B01J 20/3085; C07F 9/4056; C02F 2101/10; C08L 39/08; C08L 33/02; C02F 1/285; C02F 1/56; C07F 9/4021

(57) ABSTRACT

Complexes including a solid support and a material with a matrix structure containing domains complexing rare earth or strategic metals, preparation process thereof and use thereof for extracting or separating the rare earth or strategic metals in an aqueous or organic medium.

The present invention relates to complexes comprising a material with a matrix structure containing domains complexing rare earths or strategic metals, preparation process thereof and use thereof for extracting or separating the rare earths or strategic metals in an aqueous or organic medium.

The strategic metals are metals that are considered to be critical for the development of European industry, in particular for new technology industries. Although there is no official list of the strategic metals, antimony, indium, beryllium, magnesium, cobalt, niobium, the platinoids, gallium, germanium, tantalum, tungsten, molybdenum, titanium, mercury, cesium, lithium, strontium, and the rare earths, in particular the lanthanides, are generally considered to be strategic metals.

Due to their very specific electronic and optical properties, rare earths are indispensable elements for the new technology industries, in particular for the electronics, automotive, clean energy, aeronautics sectors and also for the defense industry. Although, despite the common assumption, the rare earths are relatively widespread in the earth's crust, the extraction of rare earths remains very expensive and inefficient due to their low concentration in mineral deposits and the fact that it is difficult to separate them from one another.

In recent years, a significant increase in the industrial demand for the strategic metals, in particular for the rare earths, has necessitated the development of new methods that are more efficient and more specific for their extraction or their recycling.

Currently, only the “liquid-liquid” extraction methods have been implemented on an industrial scale. However, due to the use in large quantities of solvents containing extractants of the rare earths, these methods have a number of drawbacks, such as generating large volumes of used solvents which are a danger to the environment, the formation of an emulsion at the interface between the aqueous phase and the organic phase, which necessitates separation by centrifugation, low selectivity between the different rare earths and between the rare earths and other metals that are inevitably present in the earth's crust or in industrial wastes, and the progressive loss of the extractants initially contained in the solvent.

In this context, several “solid-liquid” approaches to the extraction of strategic metals, in particular of the rare earths, have been developed over the last few years.

Yilmaz and Memon (Sorbents, 2009, Vol. 285-333), Alexandratos and Natesan (Macromolecules, 2001, 34, 206-210) describe respectively resins subsequently functionalized by calixarenes and use thereof for absorbing heavy metals or rare earths.

Beer et al. (J. Chem Soc., Dalton Trans., 1998, 2783-2785) describe S NH₂ Tantagel® resins functionalized by calix[4]arenes substituted by 1-acid 3 diethyl amide, the latter being grafted on resins by means of the functional group: —O—CH₂—CH₂—NH₂.

Pathak and Rao (Analytica Chimica Acta., 1996, vol. 335, no. 3, 283-290) also describe a resin based on a styrene-divinylbenzene copolymer functionalized by calixarenes (p-tert-butylcalix[8]arene).

U.S. Pat. No. 6,342,634 describes soluble acid amides, in particular acid amides in which two calixarenes are linked by a diamine, and styrene-divinylbenzene copolymer resins physisorbing said acid amides.

The materials described in these prior documents all consist of polymers previously cross-linked before grafting of the calixarenes on the surface of said polymers. The calixarenes, as a complexing domain, are not distributed homogeneously within the material and the majority remain at the surface of the material.

Garcia et al. describe polymers with ionic imprints for the “solid-liquid” extraction of lanthanides (Separation Science and Technology, 2002, vol 37, 2839-2857). The drawback of this method is the deformation of the preformed molecular cages and the loss of stability of the extractants.

EP 1 481 402 describes a method making it possible to separate the metals, such as ⁹⁰Y et ⁹⁰Sr, in an aqueous solution, using an ion exchanger comprising a carbon or graphite substrate impregnated with a hydrophobic chelating extractant having different affinities, at a selective pH, for the metals to be separated. However, the regeneration of such an exchanger can only be carried out under acidic conditions with the application of large volumes of acid solutions, which is damaging to the environment.

US 2009/0093664 describes a carbon nanotube, on which extractants of the lanthanides or of the actinides are covalently bonded, for the recovery and the separation of lanthanides and of actinides. As a result of the low rate of the fluid for passing through the matrix, this method lacks rapidity and is therefore incompatible with industrial use.

International application WO 2013/124831 describes a complex for extracting cesium from a contaminated solution, said complex comprising a porous conductive material on the surface of which calixarene groups are covalently grafted, in particular calixarene-crown ethers. It is found that said complex only allows the extraction of cesium and strontium and cannot be utilized for the extraction of rare earths. Moreover, the extraction capacity of this complex is limited to a low concentration of the metals to be extracted, due to the deformation on electrochemical grafting of the cavities for the metals of interest close to the support, the too high density of the grafted calixarenes, and the hydrophobic nature of the complexing film, which prevents access of the metals of interest to the calixarenes close to the support.

There is therefore an urgent industrial need for the development of new materials capable of being used for the extraction of the strategic metals, in particular the rare earths.

The Inventors have successfully synthesized novel materials capable of overcoming the technical defects of the prior art.

Said materials can form a thick layer up to 1 micron, while electrochemical grafting, as described previously in international application WO 2013/124831, only allows a layer of calixarenes of a maximum of 100 nm to be deposited on a support. Furthermore, the complexing cavities in a material according to the invention are distributed homogeneously in 3 dimensions, which makes it possible i) to make available a larger quantity of cage molecules (and therefore a larger capacity for capturing the metals), and ii) for the cavities, even at depth, to be accessible to the metals of interest. The specific surface of a material of the invention can be up to 1000 times greater than that of the resins functionalized by calixarenes after cross-linking thereof. As a result of its very high specific surface, the extraction time of rare earths by a material of the invention is considerably reduced with respect to the resins conventionally used for the extraction of rare earths. These specific physico-chemical properties confer on said materials a high capacity for the retention of the metals of interest and consequently allow said materials to be used on an industrial scale for the extraction and/or the separation of the different strategic metals, in particular the different rare earths.

A subject of the present invention is a novel complex comprising (i) a solid support and (ii) a solid material with a homogeneous matrix structure, having a structuring element and a cross-linking element, said material being at least insoluble in water or in an organic solvent and capable of being swelled by water or said organic solvent, at least one of the two elements bearing or forming a complexing domain of strategic metals constituted by the rare earths, antimony, indium, beryllium, magnesium, cobalt, niobium, the platinoids, gallium, germanium, tantalum, tungsten, molybdenum, titanium, mercury, cesium, lithium and strontium,
said structuring element and said cross-linking element being formed, independently of one another, by at least one entity selected from the group consisting of:
   (a) a polymer constituted by monomers, said polymer optionally bearing at least one coordinating group, advantageously a phosphate group,
   (b) a cage molecule,
   (c) a linear or branched (C₁-C₁₅) alkyl, optionally substituted by at least one substituent selected from:
      a (C₁-C₅) alkoxy group,
      a carbonyl group,
      an aryl group or a substituted aryl group, such as a tosyl group, a diazonium group,
      an aromatic heterocycle such as a pyrrolyl, furyl, thienyl or pyridinyl group,
      a sulphur atom, a sulphate or sulphonate group,
      a phosphate group,
   (d) a linear or branched (C₂-C₁₅) alkenyl or alkynyl, optionally substituted by at least one substituent selected from:
      a (C₁-C₅) alkoxy group,
      a carbonyl group,
      an aryl group or a substituted aryl group, such as a tosyl, a diazonium group,
      an aromatic heterocycle such as a pyrrolyl, furyl, thienyl or pyridinyl group,
      a sulphur atom, a sulphate or sulphonate group,
      a phosphate group, and
   (e) an aryl(C₁-C₁₅)alkyl group, optionally substituted by at least one substituent selected from:
      a (C₁-C₅) alkoxy group,
      a carbonyl group,
      an aryl group or a substituted aryl group, such as a tosyl, a diazonium group,
      an aromatic heterocycle such as a pyrrolyl, furyl, thienyl or pyridinyl group,
      a sulphur atom, a sulphate or sulphonate group,
      a phosphate group,
said cross-linking element being cross-linked with said structuring element by one or more bonds selected from the group consisting of: —C(═O)NH— NH—, —C(═O)—, —C—S—, —C—N—, —S—, —SO₃—, —C—C—, —C—O—,
   provided that, when said complexing domain is not formed by a cage molecule, then said structuring element, or said cross-linking element or both bear at least one coordinating group, advantageously a phosphate group.

By “material with a homogeneous structure” is meant a material in which the concentration of complexing domains is constant over the entire volume of the material. By “said cross-linking element being cross-linked with said structuring element” is meant a cross-linking element being covalently bonded to a structuring element and the formation of one or more three-dimensional networks by chemical bonding.

In an advantageous embodiment of the invention, the complexing domain is a rare earth complexing agent.

By “material capable of being swelled in water or in an organic solvent” is meant a material capable of adsorbing and retaining very large quantities, even up to 1000 times the mass thereof, of water or of an organic solvent.

Contact of said material with water or an organic solvent produces rapid swelling (1-10 min).

For a person skilled in the art, this concept of swelling implies a significant increase in volume without dissolution.

The swelling property of said material is indispensable for the good performance of said material.

This swelling property makes it possible to give a three-dimensional structure to a material that is initially present in a thin film and naturally two-dimensional, and consequently makes it possible to benefit from the complexing domains within said material in their entirety.

The swelling property directly affects the accessibility of the complexing domains of said material in their entirety, and consequently the property of the complex of the invention. This property can be modulated and adjusted through the thickness of the layer of said material and the cross-linking rate.

By “structuring element” is meant a constituent of the aforesaid material forming the main structure of said material and capable of establishing physical interactions within the structure in which it is used, and having the capacity to develop structuring properties leading to textures of semi-solid or solid appearance.

By “cross-linking element” is meant a constituent of the aforesaid material linking the different parts of the structuring element.

The ratio of the molar proportion between the structuring element and the cross-linking element in said material can be from 90/10 to 50/50.

The structuring element in said material determines the main physico-chemical properties of said material.

The role of the cross-linking element in a material of the invention is to form a matrix structure and to distribute the complexing domains of the strategic metals in a homogeneous manner in three dimensions in the matrix of the aforesaid material.

Said cross-linking element can also contribute additional physicochemical properties to said material of the invention.

By “a complexing domain of strategic metals etc. in particular rare earths”, is meant a group capable of bonding to a strategic metal, in particular to a rare earth, by coordination bonding, or encapsulating, by means of its spatial conformation, a strategic metal, in particular a rare earth. By way of example, such a complexing domain can be formed by a cage molecule or a phosphate group, known for their capacity to bond specifically to rare earths.

By “cage molecule” is meant a molecule having a structure containing a cavity and capable of encapsulating an atom, an ion or another molecule inside said cavity.

By way of example of cage molecules capable of being used in the context of the invention, the calix[n]arenes may be mentioned, in which n is an integer from 4 to 100, advantageously comprised between 4 and 50, yet more advantageously between 4 and 8, in particular calix[4]arene, calix[5]arene, calix[6]arene, calix[7]arene and calix[8]arene, or the crown ethers, in particular 12-crown-4, 15-crown-5, 18-crown-6 et 21-crown-7, or the cyclodextrins, in particular α-, β- and γ-cyclodextrins.

According to the invention, when neither the structuring element nor the cross-linking element contains a cage molecule, either the structuring element, or the cross-linking element, or both elements, bear at least one coordinating group, for example a phosphate group. A person skilled in the art will know how to choose a complexing domain, based on his general knowledge, depending on the strategic metal to the extracted or to be separated.

For example, 12-crown-4 ether can form a specific complexing domain in order to separate lanthanum and europium. (Ali et al., J. Chem. Chem. Eng. 2006, volume 25, 15).

Also by way of example, the calix[4]arenes functionalized by a diglycolamide can form complexing domains in order to separate europium from americium.

According to the invention, said material can contain complexing domains of different types, such as those formed respectively by the calixarenes and the crown ethers.

In a particular embodiment, said complexing domain is a combined complexing domain, formed by at least two complexing domains bound together by nucleophilic substitution or electrophilic substitution. By way of example of an entity capable of forming a combined complexing domain after cross-linking, a calixarene-crown or a phosphorylated calixarene may be mentioned.

A combined complexing domain can produce a synergistic effect for the extraction or the separation of the strategic metals with respect to a single complexing domain.

In another particular embodiment, said material contains several complexing domains specific to the different strategic metals respectively.

By “strategic metals” is meant the metals which are essential for the economic development of a state, but which have a risk of scarcity or difficulty of supply. Within the context of the present invention, the term “strategic metal” refers to metals selected from antimony, indium, beryllium, magnesium, cobalt, niobium, the platinoids, gallium, germanium, tantalum, tungsten, molybdenum, titanium, mercury, cesium, lithium, strontium, and the rare earths.

The term “rare earths” refers to a group of chemical elements constituted by scandium (Sc), yttrium (Y), and the fifteen lanthanides, namely lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb) and lutetium (Lu).

In an advantageous embodiment, the complex of the invention comprises a solid material with a homogeneous matrix structure, having a structuring element and a cross-linking element, said material being at least insoluble in water or in an organic solvent and capable of being swelled by water or said organic solvent, at least one of the two elements bearing or forming a domain complexing rare earths.

By “polymer constituted by monomers” is meant a macromolecule characterized by the repetition of one or more types of monomers.

A polymer capable of being used within the context of the invention is an organic polymer.

According to an embodiment of the invention, the polymer corresponds to formula (A) or (B):

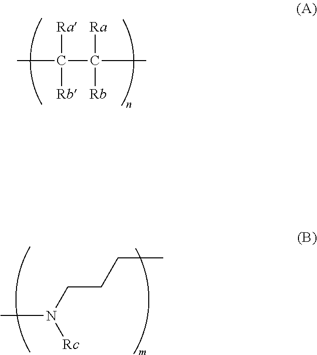

in which:

Rₐ, Rₐ′, identical or different, each represent independently of one another a hydrogen atom, a fluorine atom or a C₁-C₆ alkyl group,

R_{b}, R_{b}′ identical or different, each represent independently of one another an OH, CN, CO₂R_{d} group, a C₆-C₁₀ aryl group, or heteroaryl group with 5 or 7 members, said aryl or heteroaryl groups being optionally substituted with one or more substituents selected from an —SO₃H group and a phosphate group;

R_{c} is a hydrogen atom or —(CH₂)₂NH₂,

R_{d} is a hydrogen atom or a C₁-C₆ alkyl group.

By way of example of a polymer capable of being used in the invention as a structuring element or a cross-linking element, there may be mentioned in particular acrylic acid-based polymers (AAP), 4-vinylpyridine polymers (P4VP), fluorinated polymers such as polyvinylidine fluoride (PVDF), polyvinyl alcohols (PVA), polymethyl methacrylates (PMMA), polyethylene imines (PEI) or polyacrylonitriles (PAN).

The molecular weight of the polymer can vary over a wide range, in particular from 2,000 g·mol⁻¹ to 1,000,000 g·mol⁻¹. Preferably, the molecular weight of the polymer is comprised between 50,000 g·mol⁻¹ and 300,000 g·mol⁻¹.

In a particular embodiment, said polymer is an acrylic acid-based polymer.

By “acrylic acid-based polymer” is meant a polymer comprising the following repeat unit: —(CH₂—CX(COOH))ₙ— where X is H, or a C₁-C₆, alkyl group, in particular CH₃ or C₂H₅. Preferably, X is H. This may be a homopolymer or an acrylic acid copolymer, the latter comprising a majority of acrylic acid monomers (X═H), in particular more than 60% by weight, particularly more than 75% by weight, with respect to the total molecular weight of the copolymer.

In a preferred embodiment, the polymer forming a structuring element or a cross-linking element of a material of the invention is an acrylic acid homopolymer, also denoted AAP hereinafter, having in particular a molecular weight of 130,000 g·mol⁻¹. The solution utilized in step i) can also comprise a second acrylic acid homopolymer with a different molar weight.

In another preferred embodiment, the polymer forming a structuring element or a cross-linking element of a material of the invention is a 4-vinylpyridine-based polymer, in particular a poly(4-vinylpyridine).

By “4-vinylpyridine-based polymers” is meant the polymers originating from poly(4-vinylpyridine) by substitution of a hydrogen.

By “coordinating group” is meant a group of atoms capable of binding to a metal atom by a covalent or coordination or ionic bond, said group of atoms not being included in a ring structure, in particular a cage molecule.

The term “(C₁-C₁₅) alkyl” refers to a saturated linear or branched chain with 1 to 15 carbon atoms. Such an alkyl can be for example a methyl, ethyl, propyl, isopropyl, n-butyl, isobutyl, sec-butyl, t-butyl, pentyl, isopentyl, tert-pentyl, hexyl, heptyl, octyl, nonyl, decyl, undecyl, or a dodecyl.

The term “(C₁-C₆) alkyl” refers to a saturated linear or branched chain with 1 to 6 carbon atoms. Such an alkyl can be for example a methyl, ethyl, propyl, isopropyl, n-butyl, isobutyl, sec-butyl, t-butyl, pentyl, isopentyl, tert-pentyl, hexyl.

The term “(C₂-C₁₅) alkenyl” refers to a saturated linear or branched chain with 2 to 15 carbon atoms containing at least one double bond. Such an alkenyl can be for example a propenyl, 2-butenyl, 3-butenyl, 2-pentanyl, 4-pentenyl or 2-hexenyl.

The term “(C₂-C₁₅) alkynyl” refers to a saturated linear or branched chain with 2 to 15 carbon atoms containing at least one triple bond. By way of example, ethynyl or propynyl can be mentioned.

The term “(C₁-C₅) alkoxy” refers to a (C₁-C₅) O-alkyl radical. Within the meaning of the invention, such an alkoxy can be for example a methoxy, ethoxy, propoxy, butoxy or a pentoxy.

The term “aryl” or “C₆-C₁₀ aryl” refers to an optionally substituted monocyclic or polycyclic aromatic ring. Within the context of the invention, an aryl can be for example a phenyl, benzyl, tolyl, xylyl or a naphthyl.

Within the meaning of the invention, by “aromatic heterocycle” or “heteroaryl”, is meant an unsaturated monocyclic or polycyclic structure containing atoms of at least two different elements selected from carbon, nitrogen and sulphur atoms. By way of example of aromatic heterocyles, a pyrrolyl, furyl, thienyl, or a pyridinyl can be mentioned.

By aryl(C₁-C₁₅)alkyl is meant an aryl substituted by a (C₁-C₁₅) alkyl, in particular an aryl substituted by a methyl, ethyl, propyl, isopropyl, n-butyl, isobutyl, sec-butyl, t-butyl, pentyl, isopentyl, tert-pentyl, hexyl, heptyl, octyl, nonyl, decyl, undecyl, or a dodecyl.

The term “carbonyl” refers in particular to an anhydride group or a carboxylic group and derivatives thereof.

Within the meaning of the invention, said structuring element and said cross-linking element can be formed by compounds of the same chemical nature.

By way of example, said structuring element and said cross-linking element can both be formed by polymers, provided that either the polymer forming said structuring element, or the polymer forming said cross-linking element, or both polymers bear at least one coordinating group, such as a phosphate group.

Also by way of example, said structuring element and said cross-linking element can both be formed by cage molecules.

According to a particular embodiment of the complex of the invention, the aforesaid structuring element and the aforesaid cross-linking element are formed by an entity selected from (a) a polymer constituted by monomers and (b) a cage molecule, said structuring element being different from said cross-linking element.

In this embodiment, said polymer constituted by monomers is preferably selected from an acrylic acid-based polymer, in particular an acrylic acid homopolymer, or a polymer based on 4-vinylpyridine, in particular a poly(4-vinylpyridine).

Said cage molecule in the aforesaid embodiment is selected from the group comprising the calix[n]arenes in which n is an integer from 4 to 100, advantageously from 4 to 50, preferentially from 4 to 8, the crown ethers and the cyclodextrins.

A particular embodiment of the invention relates to a complex comprising a solid material with a homogeneous matrix structure, said material being at least insoluble in water or in an organic solvent and capable of being swelled by water or said organic solvent, in which:
the aforesaid structuring element is formed by a calix[n]arene in which n is an integer from 4 to 100, advantageously from 4 to 50, preferentially from 4 to 8 and
the aforesaid structuring element is formed by a poly(4-vinylpyridine).

Another particular embodiment of the invention relates to a complex comprising a solid material with a homogeneous matrix structure, said material being at least insoluble in water or in an organic solvent and capable of being swelled by water or said organic solvent, in which:
the aforesaid structuring element is formed by a poly(4-vinylpyridine) and
the aforesaid cross-linking element is formed by a calix[n]arene in which n is an integer from 4 to 100, advantageously from 4 to 50, preferentially from 4 to 8.

In another particular embodiment, the invention relates to a complex comprising a solid material with a homogeneous matrix structure, said material being at least insoluble in water or in an organic solvent and capable of being swelled by water or said organic solvent, in which:
the aforesaid structuring element is formed by a calix[n]arene in which n is an integer from 4 to 100, advantageously from 4 to 50, preferentially from 4 to 8 and
the aforesaid cross-linking element is formed by an acrylic acid homopolymer.

In another particular embodiment, the invention relates to a complex comprising a solid material with a homogeneous matrix structure, said material being at least insoluble in water or in an organic solvent and capable of being swelled by water or said organic solvent, in which:
the aforesaid structuring element is formed by an acrylic acid homopolymer and
the aforesaid cross-linking element is formed by a calix[n]arene in which n is an integer from 4 to 100, advantageously from 4 to 50, preferentially from 4 to 8.

The aforesaid material is deposited on the surface of a solid support and bound to the support by a non-covalent bond.

Within the meaning of the invention, said solid support can be an organic or inorganic support, in particular conductive, semi-conductive or insulating. It can be selected in particular from the metals such as copper, nickel, stainless steel, aluminium, iron, titanium, or oxides thereof, such as titanium dioxide (TiO₂), iron oxides or aluminium oxides, mineral oxides, in particular those based on silicon oxide commonly called glasses; plastics, cellulose papers, synthetic papers such as Teslin®, carbon fibres, in particular woven or non-woven and the composite materials such as glass fibre-reinforced epoxy resins, carbon fibres or natural fibres.

Depending on the different cross-linking conditions, the bond formed between the aforesaid material and the support is a non-covalent bond.

In an embodiment, the invention relates to a complex comprising or constituted by:

(i) a carbon fibre support, and

(ii) a solid material with a homogeneous matrix structure, said material being at least insoluble in water or in an organic solvent and capable of being swelled by water or said organic solvent, in which the aforesaid structuring element and the aforesaid cross-linking element are formed by an entity selected from (a) a polymer constituted by monomers and (b) a cage molecule, said structuring element being different from said cross-linking element.

A conductive support, in particular a carbon fibre support, makes it possible to electrically regenerate the aforesaid non conductive solid material and to dispense with the use of regeneration reagents, such as an acid, a base, or an organic solvent, present in a washing solution for eluting the strategic metals captured by said material, which allows the complex of the invention to be regenerated under environmentally-friendly conditions.

In a particular embodiment, a complex of the invention comprises or is constituted by a carbon fibre support and a solid material with a homogeneous matrix structure, said material being at least insoluble in water or in an organic solvent and capable of being swelled by water or said organic solvent, in which:
the aforesaid structuring element is formed by a calix[n]arene in which n is an integer from 4 to 100, advantageously from 4 to 50, preferentially from 4 to 8;
the aforesaid cross-linking element is formed by a poly(4-vinylpyridine).

In a particular embodiment, a complex of the invention comprises or is constituted by a carbon fibre support and a solid material with a homogeneous matrix structure, said material being at least insoluble in water or in an organic solvent and capable of being swelled by water or said organic solvent, in which:
the aforesaid structuring element is formed by a poly(4-vinylpyridine);
the aforesaid cross-linking element is formed by a calix[n]arene in which n is an integer from 4 to 100, advantageously from 4 to 50, preferentially from 4 to 8.

In a particular embodiment, a complex of the invention comprises or is constituted by a carbon fibre support and a solid material with a homogeneous matrix structure, said material being at least insoluble in water or in an organic solvent and capable of being swelled by water or said organic solvent, in which:
the aforesaid structuring element is formed by a calix[n]arene in which n is an integer from 4 to 100, advantageously from 4 to 50, preferentially from 4 to 8;
the aforesaid cross-linking element is formed by an acrylic acid homopolymer.

In another particular embodiment, a complex of the invention comprises or is constituted by a carbon fibre support and a solid material with a homogeneous matrix structure, said material being at least insoluble in water or in an organic solvent and capable of being swelled by water or said organic solvent, in which:
the aforesaid structuring element is formed by an acrylic acid homopolymer;
the aforesaid cross-linking element is formed by a calix[n]arene in which n is an integer from 4 to 100, advantageously from 4 to 50, preferentially from 4 to 8.

Another aspect of the invention relates to the cross-linking or structuring elements constituted by novel phosphorylated calixarenes of formula (I)

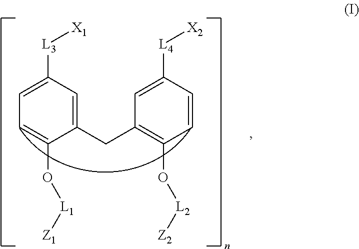

in which:
X₁ and X₂ each represent, independently of one another, H or a

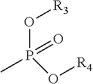

group, in which R₃ and R₄ each represent, independently of one another, H or a (C₁-C₈) alkyl group, provided that X₁ and X₂ do not simultaneously represent H,
L₁, L₂, L₃ and L₄ are spacer groups, selected independently of one another from the group consisting of a (C₃-C₁₀) cycloalkylenyl group, O, NH, —(CH₂)_{q}—, q being an integer from 0 to 12, or from 1 to 12
Z₁ et Z₂ each represent, independently of one another, a functional group selected from an optionally protected amine group, F, Cl, Br, I, OH, C(═O)H, C(═O)Hal, an aryl group or a substituted aryl group, such as a tosyl, a diazonium group, an aromatic heterocycle such as a pyrrolyl, furyl, thienyl or pyridinyl group, an optionally protected sulphate or sulphonate group, or a

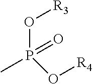

group, in which R₃ et R₄ are as defined above, Z₁ et Z₂ not both being the

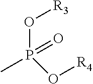

group,
n is an integer from 4 to 100, advantageously from 4 to 50, preferentially from 4 to 8.

These phosphorylated calixarenes have an improved affinity for the rare earths, in particular europium, compared with the non-phosphorylated calixarenes or other phosphorylated compounds.

Furthermore, the novel phosphorylated calixarenes of the invention bearing functional groups are capable of reacting with another compound also bearing at least one compatible functional group, which makes it possible for the aforesaid novel calixarenes to cross-link with said compounds bearing at least one compatible functional group.

In a particular embodiment, said calixarene is the compound of formula (Ia):

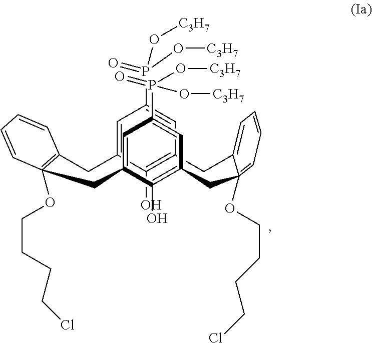

or the compound of formula (Ib):

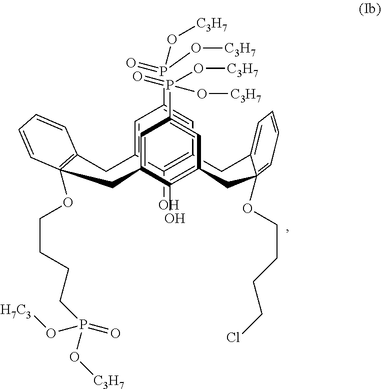

or a mixture thereof.

In a particular embodiment, the invention relates to a complex as described above, in which the aforesaid structuring element and the aforesaid cross-linking element are formed respectively by an entity selected from (a) a polymer constituted by monomers and (b) a phosphorylated calixarene of formula (I), said structuring element being different from said cross-linking element.

In a particular embodiment, the invention relates to a complex as described above, in which the aforesaid structuring element and the aforesaid cross-linking element are formed respectively by (a) an acrylic acid-based polymer, in particular an acrylic acid homopolymer, or 4-vinylpiridine-based polymer, in particular a poly(4-vinylpyridine) and (b) a phosphorylated calixarene of formula (I), said structuring element being different from said cross-linking element.

In a more particular embodiment, the invention relates to a complex as described above, in which the aforesaid structuring element and the aforesaid cross-linking element are formed respectively by an entity selected from (a) a polymer constituted by monomers and (b) the phosphorylated calixarenes of formula (Ia) and/or of formula (Ib), said structuring element being different from said cross-linking element.

In another more particular embodiment, the invention relates to a complex such as described above, in which the aforesaid structuring element and the aforesaid cross-linking element are formed respectively by an entity selected from (a) a poly(4-vinylpyridine) and (b) the phosphorylated calixarenes of formula (Ia) and of formula (Ib), said structuring element being different from said cross-linking element.

In an advantageous embodiment, the invention relates to a complex comprising a carbon fibre support and a solid material as described above, in which the aforesaid structuring element and the aforesaid cross-linking element are formed respectively by an entity selected from (a) a poly(4-vinylpyridine) and (b) the phosphorylated calixarenes of formula (Ia) and of formula (Ib), said structuring element being different from said cross-linking element.

The present invention also relates to the use of a phosphorylated calixarene of formula I, in particular the calixarenes of formula (Ia) and of formula (Ib), for the preparation of a complex of the invention.

A subject of the present invention is also a method for the preparation of an aforesaid complex of the invention.

Said method comprises:
a first step of bringing a liquid mixture or a solid mixture, comprising an agent capable of structuring and an agent capable of cross-linking, into contact with a surface of a solid support, at least one of the two agents bearing a complexing domain or being capable of forming a complexing domain after cross-linking, for strategic metals constituted by the rare earths, antimony, indium, beryllium, magnesium, cobalt, niobium, the platinoids, gallium, germanium, tantalum, tungsten, molybdenum, titanium, mercury, cesium, lithium and strontium,
   said agent capable of structuring and said agent capable of cross-linking being identical or different and corresponding to formula R₁-L-R₂, in which:

(i) L is selected from the group consisting of:
(a) a polymer, optionally substituted by at least one functional group selected from:
   a coordinating group, advantageously a phosphate group,
   an amine, optionally protected by an amine protective group,
   a halogen selected from F, Cl, Br, I,
   —OH,
   —C(═O)H,
   —C(═O)Hal in which Hal represents a halogen atom as defined above,
   a tosyl group,
   a sulphate or sulphonate group, optionally protected by a protective group of the sulphate or sulphonate group,
   a thiol, optionally protected by a protective group of the thiols
(b) a cage molecule, optionally substituted by at least one functional group selected from:
   a phosphate group,
   an amine, optionally protected by an amine protective group,
   a halogen selected from F, Cl, Br, I,
   —OH,
   —C(═O)H,
   —C(═O)Hal in which Hal represents a halogen atom,
   a tosyl group,
   a sulphate or sulphonate group, optionally protected by a protective group of the sulphate or sulphonate group,
(c) a linear or branched (C₁-C₁₅) alkyl, optionally substituted by at least one functional group selected from:
   an alkoxy group,
   a carbonyl group, such as —C(═O)H and —C(═O)Hal in which Hal represents a halogen atom,
   an aryl group or a substituted aryl group, such as a tosyl group, a diazonium group,
   an aromatic heterocycle such as a pyrrolyl, furyl, thienyl, pyridinyl group,
   a sulphate or sulphonate group, optionally protected by a protective group of the sulphate or sulphonate group,
   an amine, optionally protected by an amine protective group,
   a halogen selected from F, Cl, Br, I,
   —OH,
   a phosphate group,
   a thiol, optionally protected by a protective group of the thiols
(d) a linear or branched (C₂-C₁₅) alkenyl, optionally substituted by at least one substituent selected from:
   an alkoxyl group,
   a carbonyl group, such as —C(═O)H and —C(═O)Hal in which Hal represents a halogen atom,
   an aryl group or a substituted aryl group, such as a tosyl group, a diazonium group,
   an aromatic heterocycle such as a pyrrolyl, furyl, thienyl or pyridinyl group,
   a sulphate or sulphonate group, optionally protected by a protective group of the sulphate or sulphonate group,
   an amine, optionally protected by an amine protective group,
   a thiol, optionally protected by a protective group of the thiols,
   a halogen selected from F, Cl, Br, I,
   —OH,
   a phosphate,
(e) a linear or branched aryl(C₁-C₁₅)alkyl, optionally substituted by at least one functional group, preferentially at least 2 functional groups, selected from:
   an alkoxy group,
   a carbonyl group, such as —C(═O)H and —C(═O)Hal in which Hal represents a halogen atom
   an aryl group or a substituted aryl group, such as a tosyl group, a diazonium group,
   an aromatic heterocycle such as a pyrrolyl, furyl, thienyl, pyridinyl group,
   a sulphate or sulphonate group, optionally protected by a protective group of the sulphate or sulphonate group,
   an amine, optionally protected by an amine protective group,
   a halogen selected from F, Cl, Br, I,
   —OH,
   a phosphate,
   a thiol, optionally protected by a protective group of the thiols

(ii) R₁ and R₂ being functional groups selected from the group comprising:
an amine, optionally protected by an amine protective group,
a halogen selected from F, Cl, Br, I,
—OH,
—C(═O)H, —C(═O)Hal,
a tosyl group or an aryldiazonium group,
a sulphate or sulphonate group, optionally protected by a sulphate or sulphonate protective group,
a thiol, optionally protected by a protective group of the thiols,
a hydrogen,
   the R₁, R₂ functional groups borne by L of an agent capable of cross-linking being selected so that they are capable of reacting with the R₁, R₂ groups or the functional groups borne by L of an agent capable of structuring so as to allow cross-linking between said structuring and cross-linking agents; and
a second step of formation of a solid material with a homogeneous matrix structure, having a structuring element and a cross-linking element as defined above, by heat treatment, at a temperature comprised between 20° C. and 200° C., of an above-mentioned liquid or solid mixture comprising an agent capable of structuring and an agent capable of cross-linking on an aforesaid support.

According to the invention, the heat treatment is carried out at a temperature from 20° C. to 200° C., in particular at a temperature from 60° C. à 150° C., particularly at a temperature from 80° C. à 100° C.

The heat treatment can be carried out by any means known to a person skilled in the art, for example by heating in an oven, or by applying hot dry air directly to a liquid or solid mixture comprising an agent capable of structuring and an agent capable of cross-linking, or by applying it to supports having a high thermal conductivity, such as metals, and certain thermostable polymers such as polyimide, poly(p-phenyleneterephtalamide) (PPD-T or Kevlar®) or polytetrafluoroethylene (PTFE or Teflon®).

The duration of the heat treatment depends on the temperature applied during this treatment. A low-temperature heat treatment can take several hours. Typically, when this treatment is carried out at 80° C., the duration of the treatment can take up to 72 hours; when the temperature of the treatment is increased to 200° C., the duration of the treatment can be reduced to 60 minutes, typically from 2 to 30 minutes.

According to the invention, after the cross-linking has been initiated by the heat treatment, an agent capable of structuring and an agent capable of cross-linking become respectively the structuring element and the cross-linking element of a material as described above.

The cross-linking between the agent capable of structuring and the agent capable of cross-linking is carried out via functional groups carried respectively by the agent capable of structuring and by the agent capable of cross-linking.

A person skilled in the art will know how to choose the corresponding functional groups for an agent capable of structuring and for an agent capable of cross-linking so that they are able to react together.

By way of example, an agent capable of cross-linking bearing a halogen atom as functional group can react with an agent capable of structuring bearing a pyridinyl group, for example a poly(4-vinylpyridine).

In order to avoid undesirable reactions during the preparation of the material and control the speed of cross-linking, certain functional groups, such as the amines, sulphates or sulphonates, thiols, can be protected by the appropriate protective groups just before the heat treatment.

A person skilled in the art will know how to choose a protective group according to the functional group to be protected, the reaction conditions and the speed of the reaction.

By way of example, there may be mentioned as an amine protective group: tert-butyloxycarbonyl, 9-fluorenylmethyloxycarbonyl, carboxybenzyl, acetyl, benzoyl, benzyle or the carbamates; as sulphate or sulphonate protective group, trifluoroethyl, and as thiol protective group, an acetyl group, tert-butyl, benzyl, 2-cyanoethyl or a disulphide bridge may be mentioned.

These protected functional groups are deprotected according to conventional methods before the heat treatment.

Said liquid mixture comprising an agent capable of structuring and an agent capable of cross-linking can also comprise a solvent.

Said solvent can be an inorganic or organic solvent making it possible to solubilize said agent capable of structuring and said agent capable of cross-linking in order to obtain a clear and homogeneous solution. A person skilled in the art will know how to choose a suitable solvent for solubilizing said agent capable of structuring or said agent capable of cross-linking. By way of example, an acrylic acid-based polymer can be solubilized in an alcohol, in particular ethanol, or a hydroalcoholic mixture; P4VP can be solubilized in an alcohol or tetrahydrofuran (THF); the calixarenes can be solubilized in an organic solvent, such as THF or DMSO (dimethyl sulphoxide).

When the heat treatment is carried out on a solid mixture comprising an agent capable of structuring and an agent capable of cross-linking, said mixture can be obtained from an aforesaid liquid mixture after removal of the solvent.

Removal of the solvent can be carried out by any conventional techniques known to a person skilled in the art, such as simple drying in air, in particular for the liquid mixtures comprising alcoholic solvents, for example with ethanol, or also evaporation under reduced pressure and/or by heating for the solvents having a higher boiling point, in particular for the liquid mixtures comprising hydroalcoholic solvents.

The thickness of the aforesaid material can be adjusted, according to the general knowledge of a person skilled in the art, by means of the respective concentrations of the agent capable of structuring and of the agent capable of cross-linking and/or by means of the successive deposition of layers of material on another layer previously deposited.

The process of the invention can also comprise, after the formation of said solid material as previously defined in the invention, a step of rinsing of said obtained material with water or with a suitable solvent.

Rinsing makes it possible to remove all the unreacted products after the step of forming the material as previously defined in the invention.

A liquid mixture can be applied to the surface of the solid support by different methods, in particular by immersion/extraction, centrifuging (spin coater), sprinkling (spray), spraying (ink jet, pistol spray) or by transfer (paintbrush, felt brush, pad).

By “a solid mixture” is meant a mixture obtained from an initial liquid mixture after evaporation of the solvent in said liquid mixture. The agent capable of structuring and the agent capable of cross-linking are not, or only slightly, cross-linked together in said solid mixture.

Said solid mixture can be formed in situ on a solid support after removal of the solvent.

Said solid mixture can also be formed beforehand on a support different from that contained in the complex of the invention and subsequently deposited on the support contained in the complex.

In a particular embodiment, the invention relates to a method for the preparation of a complex comprising a solid material as previously defined in the invention, in which said structuring element and said cross-linking element are selected from (a) a polymer constituted by monomers and (b) a cage molecule, said structuring element and said cross-linking element being different, said method comprising the formation of said material by heat treatment, at a temperature comprised between 20° C. et 200° C., of a liquid or solid mixture comprising:

(i) a polymer constituted by monomers, optionally substituted by at least one functional group selected from:
a coordinating group, advantageously a phosphate,
an amine, optionally protected by an amine protective group,
a halogen selected from F, Cl, Br, I,
—OH,
—C(═O)H,
—C(═O)Hal in which Hal represents a halogen atom,
a tosyl group,
a sulphate or sulphonate group, optionally protected by a protective group of the sulphate or sulphonate group,
a thiol, optionally protected by a protective group of the thiols, and

(ii) a cage molecule, optionally substituted by at least one functional group selected from:
a phosphate group,
an amine, optionally protected by an amine protective group,
a halogen selected from F, Cl, Br, I,
—OH,
—C(═O)H,
—C(═O)Hal in which Hal represents a halogen atom,
a tosyl group,
a sulphate or sulphonate group, optionally protected by a protective group of the sulphate or sulphonate group,
a thiol, optionally protected by a protective group of the thiols
   said polymer and said cage molecule being capable of reacting by means of functional groups so as to allow cross-linking between said polymer and said cage molecule.

In a more particular embodiment, the invention relates to a method for the preparation of a complex comprising a solid material as previously defined in the invention, in which said structuring element and said cross-linking element are selected from a poly(4-vinylpyridine) and a calix[n]arene in which n is an integer from 4 to 100, said structuring element and said cross-linking element being different, said method comprising the formation of said material by heat treatment, at a temperature comprised between 20° C. et 200° C., of a liquid or solid mixture comprising a poly(4-vinylpyridine) and a calix[n]arene substituted by a halogen.

A subject of the present invention is also a complex capable of being obtained by the implementation of the process as described above.

When a complex obtained according to the aforesaid process comprises a solid support, said material is bound to said solid support by non-covalent bonding.

Certain complexes of the present invention comprising a solid support can be obtained by any technique known to a person skilled in the art, in particular according to the following procedure:

i) bringing a surface of said solid support into contact with a solution comprising a polymer, a complexing agent, and a solvent;

said solution not comprising an adhesion primer other than said polymer;

ii) removal of the solvent from the solution in contact with said surface; and

iii) formation of the material as defined above and fixing said material on said surface by gamma or electronic radiation treatment.

The gamma radiation, in particular radiation at a wavelength comprised between 1 nm and 5 nm, can for example be produced from a cesium 137 source of 55 TBq (660 keV gamma photons) the radiation dosage of which can vary between 5.5 and 50 Gy.

An electron radiation treatment can be implemented by an electron beam originating for example from a column of a scanning electron microscope. Typically a power range from 0.5 to 30 keV is preferred.

Said polymer, said solvent and said solid support implemented in step i) are respectively as previously defined.

Said complexing agent is a compound capable of forming a complexing domain as previously defined after cross-linking with a polymer.

Said process can also comprise a step o), prior to step i), consisting of subjecting the solid support to a surface pretreatment of the oxidative type, in particular chemical and/or radiation, so as to increase the affinity of the solid support for the solution containing the polymer and the complexing agent.

The oxidative treatment can be carried out by oxygen plasma and the radiation treatment can be UV-Ozone activation.

Other complexes according to the invention, when said complexes comprise a solid support with a layer of material, in which the structuring element or the cross-linking element is formed by an acrylic acid-based polymer, can be obtained according to the following procedure:

i) bringing a surface of said solid support into contact with a solution comprising an acrylic acid-based polymer, a complexing agent, and a solvent;

said solution not comprising an adhesion primer based on aryldiazonium salts;

ii) removal of the solvent from the solution in contact with said surface; and

iii) fixing the polymer on said surface by heat treatment at a temperature between 150° C. et 300° C., more particularly at a temperature of approximately 200° C., or gamma or electronic radiation treatment.

Said acrylic acid-based polymer, said complexing agent, said solvent and said solid support implemented in step i) are respectively as previously defined.

Said process can also comprise a step o), prior to step i), consisting of subjecting the solid support to a surface pretreatment of the oxidation type, in particular chemical and/or radiation, so as to increase the affinity of the solid support for the solution containing the polymer and the complexing agent.

Another aspect of the invention relates to the use of the complexes described in the present invention, or complexes obtained by the processes described in the present description, in order to extract or separate strategic metals, in particular rare earths, from an aqueous medium or an organic liquid medium.

According to another aspect, a subject of the invention is a method for extracting or separating strategic metals, in particular rare earths, from an aqueous medium or an organic liquid medium, said method comprising a step of bringing an aqueous or organic liquid medium into contact with at least one complex described in the present invention, or complexes obtained by the processes described in the present description.

According to the invention, by “aqueous medium” or by “organic liquid medium” is meant a waste water effluent, or any solution containing strategic metals, in particular rare earths, to be extracted or separated.

By means of the complexes of the invention containing complexing domains specific to one or more particular strategic metals, said method of the invention makes it possible to extract or separate specifically one or more particular strategic metals, in an aqueous or organic liquid medium comprising several strategic metals, in particular rare earths, of the same group or of different groups.

A person skilled in the art will know how to choose a complex of the invention depending on the strategic metals to be extracted or separated and according to the properties of the medium from which the strategic metals are extracted. Specifically, a person skilled in the art will know how to choose a complex of the invention comprising a materiel as previously defined which is insoluble but capable of being swelled in the medium from which the strategic metals are extracted.

The method of the invention can also comprise a step of recovery of the strategic metals, in particular the rare earths, retained in the aforesaid complex.

According to the invention, the strategic metals, in particular the rare earths, can be recovered by the methods known to a person skilled in the art, in particular by bringing said complex retaining the strategic metals to be recovered into contact with a desorption solution, optionally by implementing an electro-desorption method.

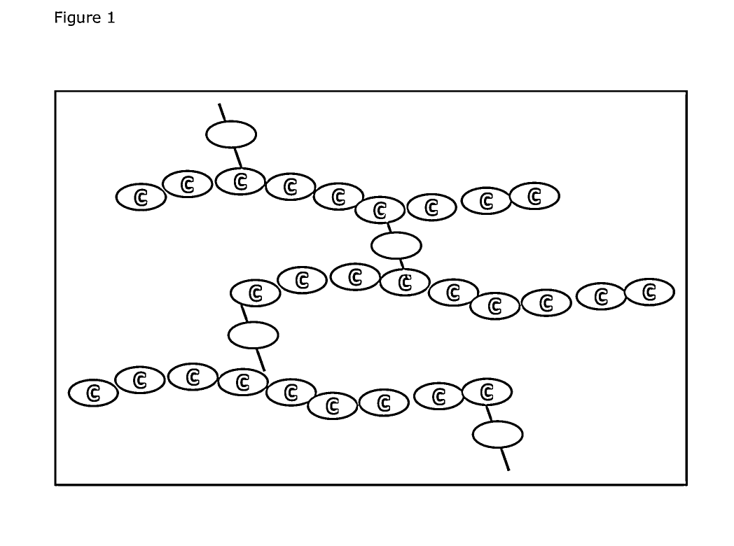

The present invention is illustrated by FIGS. 1 to 6 and the following Examples 1 to 5.

FIG. 1 shows the matrix of a material according to the invention, in which the structuring element is formed by cage molecules (C).

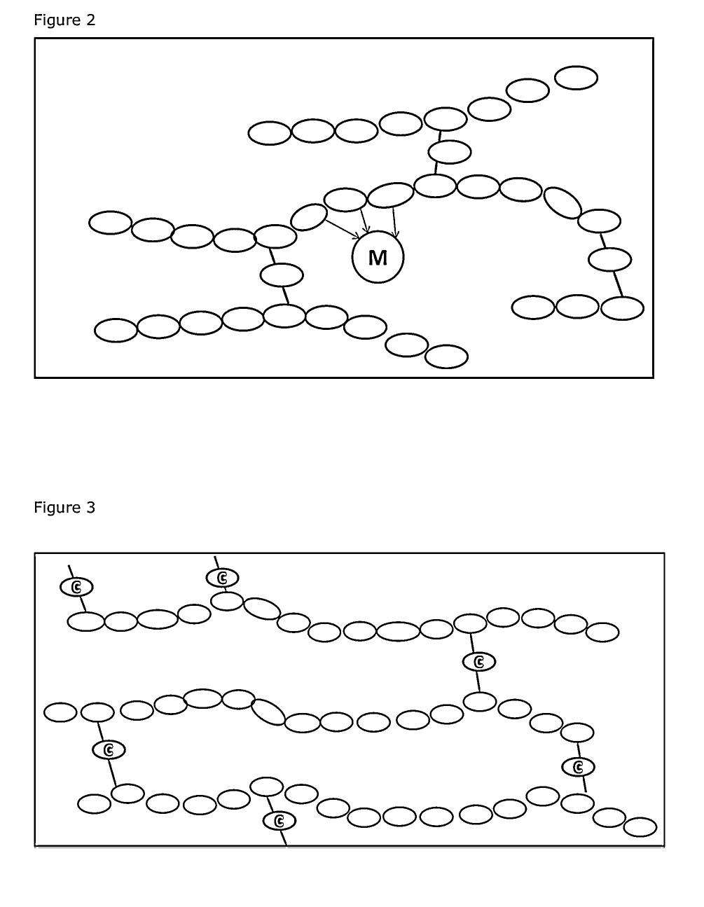

FIG. 2 shows the matrix of a material according to the invention, in which the complexing domain for a molecule of interest (M) is formed by the structuring element.

FIG. 3 shows the matrix of a material described in the invention, in which the structuring element is formed by a polymer; the cross-linking element is formed by the cage molecules.

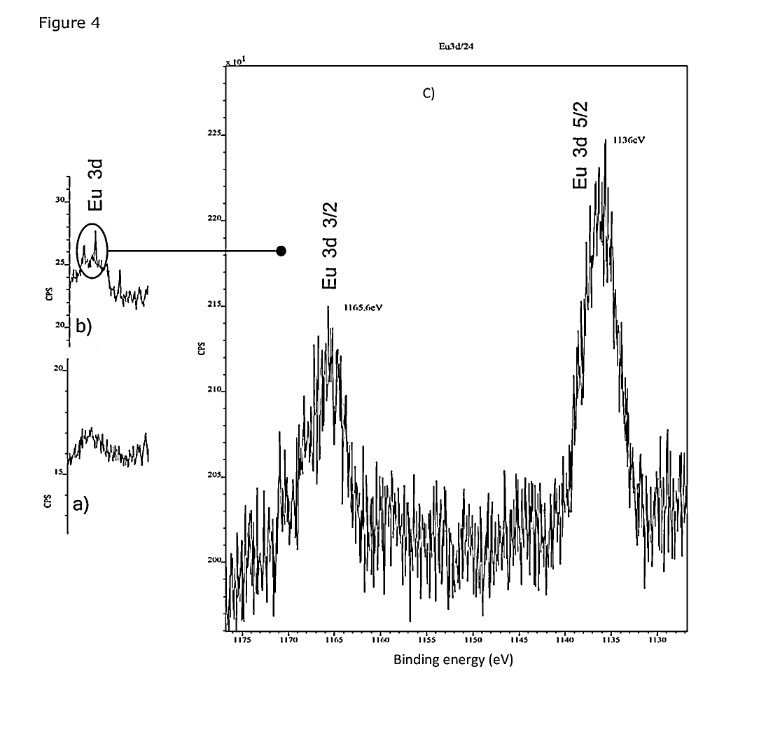

FIG. 4 shows the retention of europium by the “KT103-P4VP carbon felt” complex (b) and c)) or by a reference complex (a) as measured according to Example 2. The presence of europium is detected by X-ray photo-electron spectroscopy.

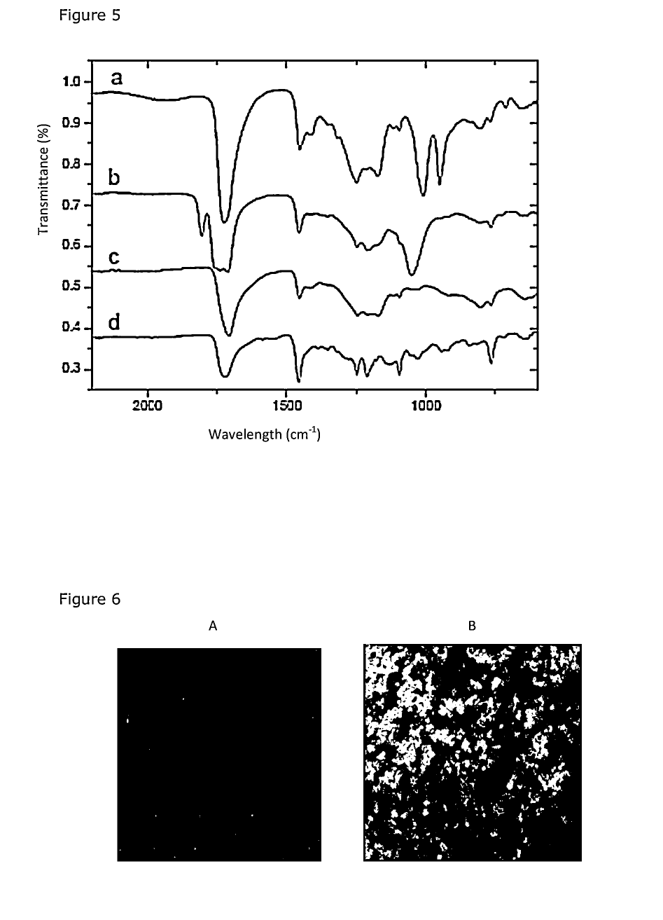

FIG. 5 shows the IR transmittance as a function of the wavelength of an “AAP-calix-gold” complex obtained by heat treatment according to Example 4. Spectrum (a) is recorded just after the deposition and drying of the alcohol. Spectrum (b) is recorded after baking at 200° C. Spectrum (c) is obtained after of hydrolysis for 10 minutes at pH10. Spectrum (d) is obtained after rapid rinsing of an initial film with water.

FIGS. 6A and 6B show a complex of the invention constituted by a carbon felt covered by a three-dimensional matrix material, before (FIG. 6A) and after (FIG. 6B) contact with water, clearly demonstrating the swelling of said material.

### EXAMPLE 1: EXTRACTION OF CESIUM BY A “KT101-P4VP-CARBON FELTS” COMPLEX

### (i) Synthesis of 1,3-alternate-diiodobutyl calix[4]arene-crown-6 (KT101)

The calixarene KT101 is synthesized according to the chemical reaction illustrated below:

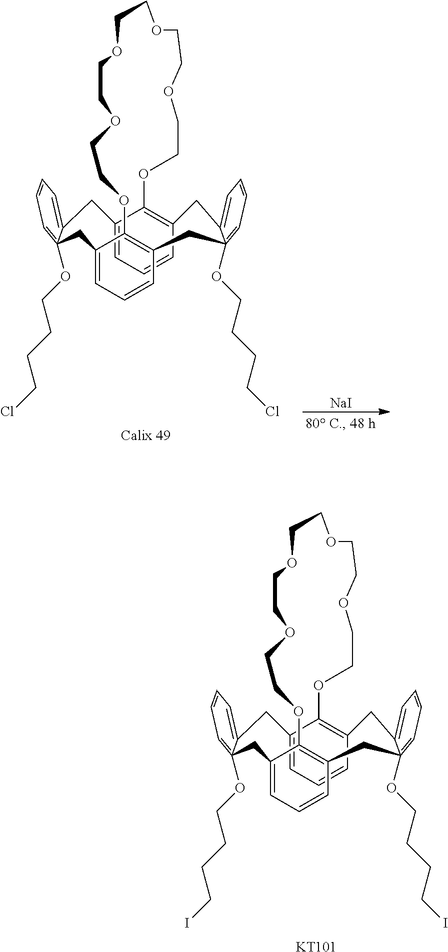

### (i) Preparation of KT101

200 mg of calixarene 49 (0.248 mmol) and 81.72 mg of NaI (0.545 mmol) are dissolved in 6 mL of 2-butanone and under stirring for 48 h at 80° C.

After the reaction, the solvent is evaporated off under vacuum. The residue obtained is extracted 3 times with 30 mL of dichloromethane. The organic layer obtained is washed once with 60 mL of salt water and then filtered through celite. 76 mg of KT101 (ESI-MS m/z 1013.19 (M+Na)⁺) are obtained in the form of white powder after filtration under vacuum.

### (ii) Preparation of the “KT101-P4VP-Carbon Felt” Complex

76 mg of KT101 and 50 mg of poly(4-vinylpyridine) (P4VP) are dissolved in 5 ml of distilled THF. The reaction mixture thus obtained is heated under reflux at 80° C. for 72 h in order to form N-calixpyridinium iodide with the formula below:

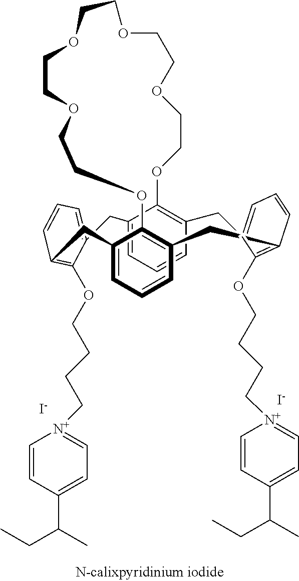

The carbon felts (Mersen®) in form of a disk with a diameter of 2 cm are immersed in said reaction mixture containing the aforesaid N-calixpyridinium iodide in a quantity of 10⁻³ M-10⁻⁴ M in order to extract the element of interest present at a concentration of 10⁻³ M-10⁻⁴ M in an aqueous solution used as a model of the effluent. They are then baked in an oven at 100° C. for 8 h in order to complete the cross-linking reaction.

At the end of this step, the carbon felts are obtained covered with P4VP polymer cross-linked with KT101.

### (iii) Treatment of Effluents Containing Cesium Salt

After rinsing with water and drying at 100° C., the “KT101-P4VP-carbon felts” complex obtained in step (ii) is subjected to a competitive test in order to assess the effectiveness and specificity of said complex for the extraction of cesium.

Aliquots of approximately 0.9 g originating from 3 disks of carbon felt obtained in step (ii) are immersed, for 20 minutes or 5 days, at ambient temperature, in a solution of 20 ml of concentrations approximately 10⁻⁴ M of cesium nitrate and approximately 0.1 M of sodium nitrate. This solid phase constituted by carbon felt loaded with polymer bearing calixarenes at a concentration that makes it possible to have an extraction percentage comprised between 10 and 90%.

The cesium concentrations, at the start (Cⁱ) and at the end of the treatment (C^{f}), are determined by atomic absorption spectrometry.

The results are illustrated in Table 1 below.

TABLE 1

| | after 20 min | after 5 days |
|---|---|---|
| Final concentration Cs after | 0.085 | 0.069 |
| treatment with P4VP-KT101, | | |
| (C^{f,} mM) | | |
| Initial concentration Cs (C^{i,} | 0.223 | 0.222 |
| mM) | | |
| % E* | 61.88 | 68.91 |

*% E is the extraction percentage of cesium determined according to the formula: % E = (Cⁱ − C^{f})/Cⁱ × 100%, in which Cⁱ and C^{f} are respectively the concentration of cesium before and after extraction.

These results show that the “KT101-P4VP-carbon felts” complex of the invention effectively and specifically extract the cesium present in a effluent.

### EXAMPLE 2: EXTRACTION OF EUROPIUM BY A “KT103-P4VP-CARBON FELTS” COMPLEX

### (i) Synthesis of a Mixture of Phosphorylated Calixarenes

Phosphorylated calixarenes are synthesized according to the following reaction steps:

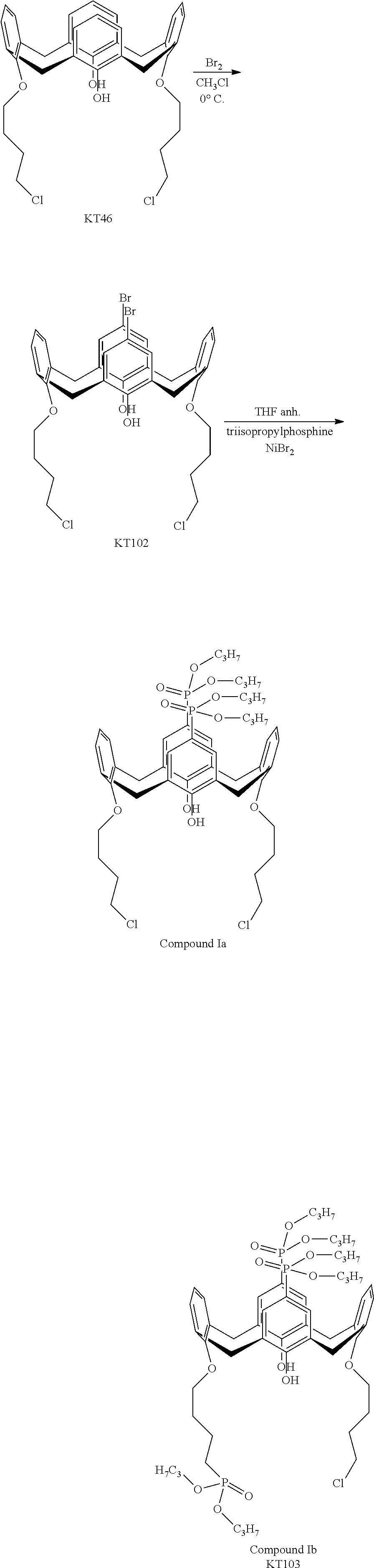

The calixarene 5,17-dibromo-25,27-bis(4-chlorobutoxy)calix[4] arene (denoted hereafter KT 102) is obtained by the direct addition of bromine atoms onto the calixarene KT46 according to the method described by Guillon et al. (Supramolecular Chemistry, 2004, 16, 319). KT 102 is then subjected to an Arbuzov reaction catalyzed by NiBr₂, in order to produce a mixture of two phosphorylated calixarenes, namely compound Ia and compound Ib, together named the KT103 series is used as it is in the following reactions without further purification.

The composition of the KT103 mixture is confirmed by MALDI-TOF spectrometry. The spectrum of said mixture shows two respective peaks at m/z=1065.20 and at m/z=1195.29, corresponding to two ionic complexes formed by two compounds of the KT103 mixture with cesium trifluoroacetate (CsTFA).

### (ii) Preparation of the “KT103-P4VP-Carbon Felt” Complex

The mixture of calixarenes KT103 and poly(4-vinylpyridine) (P4VP) is dissolved in distilled THF (5 ml of solvent for 0.5 mmol of calixarene and 1.9 mmol of P4VP). The reaction mixture thus obtained is heated under reflux at 80° C. for 72 h in order to form N-calixpyridinium chlorides.

The reaction medium is deposited by immersion as described in the preceding example on carbon felts (Mersen®) in the form of disks with a diameter of 2 cm in a sufficient quantity to extract the element of interest present at a concentration of 10⁻³ M-10⁻⁴ M in an aqueous solution used as a model of the effluent. Then the carbon felts are baked in an oven at 100° C. for 8 h in order to complete the cross-linking reaction.

After the heat treatment, the carbon felts are rinsed in deionized water for 8 h at ambient temperature in order to confirm the insolubility of the “P4VP-KT103” polymer in an aqueous medium.

### (iii) Preparation of a Reference Complex Based on P4VP

A reference complex consisting of a gold plate coated with a P4VP polymer cross-linked with diiodohexane is prepared according to the same method. The cross-linked polymer is deposited on a gold plate, washed with ethanol then with deionized water and dried in an oven for 8 h at 100° C.

### (iv) Treatment of a Solution Simulating an Effluent Containing Europium Salt

Approximately 0.9 g of carbon felts (3 disks with a diameter of 2 cm) obtained in step (ii) are immersed, for 20 minutes, at ambient temperature in 20 ml of a solution of containing approximately 10⁻³ M of europium nitrate. The carbon felts are then rinsed with ethanol in order to remove the non-complexed metallic ions and dried in an oven. The europium retained by the “KT103-P4VP-carbon felt” complex is analyzed by XPS (X-ray photoelectron spectrometry).

The comparative experiment is carried out with the reference complex prepared according to Example 2 (iii).

The respective spectra of the “KT103-P4VP-carbon felt” complex and of the reference complex are shown in FIG. 4.

Unlike the reference complex which does not absorb the europium and on which the presence of these ions is not observed, the “KT103-P4VP-carbon felt” complex retains the europium ions contained in the test solution.

### EXAMPLE 3: EXTRACTION OF THE CESIUM BY A “AAP-CALIX-CARBON FELT” COMPLEX

### (i) Preparation of the “AAP-Calix-Carbon Felt” Complex by Gamma Radiation Treatment

A solution of polyacrylic acid (AAP) is prepared with 50 mg in 10 ml of ethanol. A solution of 25,27-bis(4-chlorobutoxy)calix[4]arenes-crown-6 (calixarene 49) is prepared with 11 g in 40 ml of DMSO (dimethylsulphoxide).

The final solution is made by adding 600 μl of the solution of calix 49 in 10 ml of solution of AAP (i.e. approximately 5 mg of AAP for 16.5 mg of calix 49 per millilitre of solution). The mixture is left under stirring for 30 minutes in order to clarify it.

Disks of carbon felt (RVG 4000—0.7 m²/g—d=0.088) with a diameter of 2.8 cm (340 mg) are subjected to an Ar—O₂ [90-10%] plasma treatment for 10 minutes in order to wet them. This treatment limits recession of the liquid during drying and makes it possible to obtain coatings covering all of the fibres.

A solution of polyethylene imine (PEI), having a molecular weight of 25,000, is prepared by the dissolution of 5 mg of PEI in 10 ml of deionized water.

A first impregnation of the disks of carbon felt is carried out with an aqueous solution of polyethylene imine (PEI) at 5 mg/10 ml.

The felt is filled using a Pasteur pipette until visual detection that the impregnation is complete. Then the felt is left to dry. The primary coating of PEI (polymer having positive charges) reinforces the polyelectrolytic properties of the AAP (negatively charged) and leads to a better coating of the fibres.

A second impregnation is carried out with the solution of AAP and calixarene described above until visual detection that the impregnation is complete. The felt is left to dry naturally.

The gamma irradiation is carried out for 8 h in a gammacell 3000 Elan with 662 keV photons (5.5 Gy/min).

Repeated rinsing with water or with basic solutions (pH=9-10) do not make it possible to remove the interferential stains visible on the carbon fibres. The AAP coatings are therefore immobilized on the fibres.

The control samples (in which the calixarene 49 is not added) are prepared according to the same process.

### (ii) Treatment of a Solution Containing Cesium Salt

Analysis of the selective extraction of cesium from an aqueous solution containing chloride ions is carried out according to the following method:

3 felts prepared according to step (i), as well as the control felts coated with AAP, are introduced respectively into a pill bottle with a stopper and covered with 20 ml of a solution of 0.2 mM/10 mM of Cs+/Na+ in tap water; they are left for 20 min at ambient temperature while stirring regularly. The cesium molar concentrations of the samples was then measured by flame absorption spectroscopy.

The results are illustrated in Table 2 below.

TABLE 2

| Sample | Concentration (mmol/ml) |
|---|---|
| Initial solution | 0.255 |
| Solution after contact with “AAP- | 0.191 |
| calix-carbon felts” complex | |

The result shows that the “AAP-calix-carbon felts” complex makes it possible to extract the cesium cation.

### EXAMPLE 4: PREPARATION OF THE “AAP-CALIX-GOLD” COMPLEX BY HEAT TREATMENT

A solution of AAP (acrylic acid polymer) is prepared with 150 mg in 10 ml of ethanol. A solution of calixarene 49 is prepared with 11 g in 40 ml of DMSO (dimethylsulphoxide).

The final solution is made by adding 200 μl of the solution of calix 49 into 10 ml of AAP solution. Stirring for 30 minutes is necessary in order to clarify the mixture.

A glass slide of the microscopy object slide type (7.5×2.5 cm) is coated in gold by a vacuum metallization process.

The application of the AAP solution onto the gold-coated slide is carried out by immersion-emersion in order to obtain after evaporation of the ethanol a homogeneous film of AAP that covers well, with a thickness of 70 to 100 nm. At this stage traces of DMSO are still present in the film.

The glass slides coated with AAP are then heated at 200° C. for 30 min in a simple oven at atmospheric pressure and without specific precautions. The DMSO is removed in this step.

FIG. 5 shows the results of the IR spectra recorded on the gold-coated slides at different stages of the process.

It is sought to detect the presence of calixarene in the AAP film. On the spectrum (a) which is recorded just after the deposition and drying of the alcohol, DMSO is also present (bands 1008 and 947 cm-1).

The bands corresponding to the calixarenes are faintly visible in particular at 1454, 1246, 1208, 1093 and 764 cm-1.

Spectrum (b) is recorded after baking at 200° C. The DMSO has disappeared. The bands corresponding to the calixarenes are still visible.

Spectrum (c) is obtained after hydrolysis for 10 minutes at pH=10.

Spectrum (d) is obtained with rapid rinsing of an initial film (equivalent to spectrum (a)) with water. Partial removal of the AAP is obtained and it becomes easier to observe the bands of the calixarene molecules which are relatively hydrophobic. This procedure makes it possible to increase the proportion of calixarene in the AAP films. This spectrum also shows that a significant proportion of calixarene is present in the film. With reference to the concentrations of the initial solutions of AAP and calixarene, the proportion on the spectrum (a) is 60 mg of calixarene for 150 mg of AAP.

### EXAMPLE 5: PREPARATION OF THE “AAP-CALIX-GOLD” COMPLEX BY GAMMA RADIATION TREATMENT

A solution of AAP is prepared with 150 mg in 10 ml of ethanol.

A solution of calixarene 49 is prepared with 11 g in 40 ml of DMSO (dimethylsulphoxide).

The final solution is made by adding 200 μl of the solution of calix 49 into 10 ml of AAP solution. Stirring for 30 minutes is necessary in order to clarify the mixture.

With reference to the dry compounds this represents 60 mg of calix 49 for 150 mg of AAP per millilitre of solution.

A glass slide of the microscopy object slide type (7.5×2.5 cm) is coated in gold by a vacuum metallization process. The application of the AAP solution onto the gold-coated slide is carried out by immersion-emersion in order to obtain after evaporation of the ethanol a homogeneous covering film of AAP with a thickness of 70 to 100 nm. At this stage traces of DMSO are still present in the film; prolonged natural drying, or even heating at 100° C./1 h is sufficient to remove the traces of DMSO solvent.

The glass slide coated with AAP+calix 49 is introduced into the irradiation chamber of a gammacell 3000 Elan with 662 keV photons (5.5 Gy/min). The irradiation lasts 8 h.

The immobilization of the coating is tested by washing abundantly with water or with basic solutions (ph=9-10). The film which is initially very soluble in water or the bases becomes insoluble and immobilized after irradiation.

Finally, the AAP film is immobilized on the surface after irradiation for 8 h. Radical cross-linking mechanisms have taken place within the volume of the film.

1. Complex comprising (i) a solid support and (ii) a solid material with a homogeneous matrix structure, having a structuring element and a cross-linking element, said material being at least insoluble in water or in an organic solvent and capable of being swelled by water or said organic solvent, at least one of the two elements bearing or forming a complexing domain of strategic metals constituted by the rare earths, antimony, indium, beryllium, magnesium, cobalt, niobium, the platinoids, gallium, germanium, tantalum, tungsten, molybdenum, titanium, mercury, cesium, lithium and strontium, in particular the rare earths,
said structuring element and said cross-linking element being formed, independently of one another, by at least one entity selected from the group consisting of:
(a) a polymer constituted by monomers, said polymer optionally bearing at least one coordinating group, advantageously a phosphate group,
(b) a cage molecule,
(c) a linear or branched (C₁-C₁₅) alkyl, optionally substituted by at least one substituent selected from:
a (C₁-C₅) alkoxy group,
a carbonyl group,
an aryl group or a substituted aryl group, such as a tosyl group, a diazonium group,
an aromatic heterocycle such as a pyrrolyl, furyl, thienyl or pyridinyl group,
a sulphur atom, a sulphate or sulphonate group,
a phosphate group,
(d) a linear or branched (C₂-C₁) alkenyl or alkynyl, optionally substituted by at least one substituent selected from:
a (C₁-C₅) alkoxy group,
a carbonyl group,
an aryl group or a substituted aryl group, such as a tosyl, a diazonium group,
an aromatic heterocycle such as a pyrrolyl, furyl, thienyl or pyridinyl group,
a sulphur atom, a sulphate or sulphonate group,
a phosphate group, and
(e) an aryl(C₁-C₁₅)alkyl group, optionally substituted by at least one substituent selected from:
a (C₁-C₅) alkoxy group,
a carbonyl group,
an aryl group or a substituted aryl group, such as a tosyl, a diazonium group,
an aromatic heterocycle such as a pyrrolyl, furyl, thienyl or pyridinyl group,
a sulphur atom, a sulphate or sulphonate group,
a phosphate group,
said cross-linking element being cross-linked with said structuring element by one or more bonds selected from the group consisting of: —C(═O)NH—, —NH—, —C(═O)—, —C—S—, —C—N—, —S—, —SO₃—, —C—C—, —C—O—,
provided that, when said complexing domain is not formed by a cage molecule, then said structuring element, or said cross-linking element or both bear at least one coordinating group, advantageously a phosphate group.

2. Complex according to claim 1, wherein said structuring element and said cross-linking element are formed by an entity selected from (a) a polymer constituted by monomers and (b) a cage molecule, said structuring element being different from said cross-linking element.

3. Complex according to claim 1, wherein the polymer constituted by monomers is selected from an acrylic acid-based polymer, in particular an acrylic acid homopolymer, or a polymer based on 4-vinylpyridine, in particular a poly(4-vinylpyridine).

4. Complex according to claim 1, wherein the cage molecule is selected from the group comprising the calix[n]arenes in which n is an integer from 4 to 100, advantageously from 4 to 50, preferentially from 4 to 8, the crown ethers and the cyclodextrins.

5. Complex according to claim 1, wherein said structuring element of said complex is formed by a calix[n]arene in which n is an integer from 4 to 100, advantageously from 4 to 50, preferentially from 4 to 8, and said cross-linking element of said complex is formed by a poly(4-vinylpyridine).

6. Complex according to claim 1, wherein said structuring element of said complex is formed by a poly(4-vinylpyridine) and said cross-linking element of said complex is formed by a calix[n]arene in which n is an integer from 4 to 100, advantageously from 4 to 50, preferentially from 4 to 8.

7. Method for the preparation of a complex according to claim 1, said method comprising:
a first step of bringing a liquid mixture or a solid, comprising an agent capable of structuring and an agent capable of cross-linking, into contact with a surface of a solid support, at least one of the two agents bearing a complexing domain or being capable of forming a complexing domain after cross-linking, for strategic metals constituted by the rare earths, antimony, indium, beryllium, magnesium, cobalt, niobium, the platinoids, gallium, germanium, tantalum, tungsten, molybdenum, titanium, mercury, cesium, lithium and strontium,
said agent capable of structuring and said agent capable of cross-linking being identical or different and corresponding to formula R₁-L-R₂, in which:
(i) L is selected from the group consisting of:
(a) a polymer, optionally substituted by at least one functional group selected from:
a coordinating group, advantageously phosphate,
an amine, optionally protected by an amine protective group,
a halogen selected from F, Cl, Br, I,
—OH,
—C(═O)H,
—C(═O)Hal in which Hal represents a halogen atom,
a tosyl group,
a sulphate or sulphonate group, optionally protected by a protective group of the sulphate or sulphonate group,
a thiol, optionally protected by a protective group of the thiols
(b) a cage molecule, optionally substituted by at least one functional group selected from:
a phosphate group,
an amine, optionally protected by an amine protective group,
a halogen selected from F, Cl, Br, I,
—OH,
—C(═O)H,
—C(═O)Hal in which Hal represents a halogen atom,
a tosyl group,
a sulphate or sulphonate group, optionally protected by a protective group of the sulphate or sulphonate group,
(c) a linear or branched (C₁-C₁₅) alkyl, optionally substituted by at least one substituent selected from:
an alkoxy group,
a carbonyl group, such as —C(═O)H, and —C(═O)Hal in which Hal represents a halogen atom,
an aryl group or a substituted aryl group, such as a tosyl group, a diazonium group,
an aromatic heterocycle such as a pyrrolyl, furyl, thienyl, pyridinyl group,
a sulphate or sulphonate group, optionally protected by a protective group of the sulphate or sulphonate group,
an amine, optionally protected by an amine protective group,
a halogen selected from F, Cl, Br, I,
—OH,
a phosphate group,
a thiol, optionally protected by a protective group of the thiols
(d) a linear or branched (C₂-C₁₅) alkenyl or alkynyl, optionally substituted by at least one functional group selected from:
an alkoxy group,
a carbonyl group, such as —C(═O)H and —C(═O)Hal in which Hal represents a halogen atom,
an aryl group or a substituted aryl group, such as a tosyl group, a diazonium group,
an aromatic heterocycle such as a pyrrolyl, furyl, thienyl or pyridinyl group,
a sulphate or sulphonate group, optionally protected by a protective group of the sulphate or sulphonate group,
an amine, optionally protected by an amine protective group,
a thiol, optionally protected by a protective group of the thiols
a halogen selected from F, Cl, Br, I,
—OH,
a phosphate,
(e) a linear or branched aryl(C₁-C₁₅)alkyl, optionally substituted by at least one functional group, preferentially at least 2 functional groups, selected from:
an alkoxy group,
a carbonyl group, such as —C(═O)H, and —C(═O)Hal in which Hal represents a halogen atom
an aryl group or a substituted aryl group, such as a tosyl group, a diazonium group,
an aromatic heterocycle such as a pyrrolyl, furyl, thienyl or pyridinyl group,
a sulphate or sulphonate group, optionally protected by a protective group of the sulphate or sulphonate group,
an amine, optionally protected by an amine protective group,
a halogen selected from F, Cl, Br, I,
—OH,
a phosphate,
a thiol, optionally protected by a protective group of the thiols
(ii) R₁ and R₂ being selected from the group comprising:
an amine, optionally protected by an amine protective group,
a halogen selected from F, Cl, Br, I,
—OH,
—C(═O)H, —C(═O)Hal,
a tosyl group or an aryldiazonium group,
a sulphate or sulphonate group, optionally protected by a protective group of the sulphate or sulphonate group,
a thiol, optionally protected by a protective group of the thiols
a hydrogen,
the R₁, R₂ groups or the functional groups borne by L of an agent capable of cross-linking being selected so that they are capable of reacting with the R₁, R₂ groups or the functional groups borne by L of an agent capable of structuring so as to allow cross-linking between said structuring and cross-linking agents; and
a second step of formation of a solid material with a homogeneous matrix structure as previously defined, by heat treatment, at a temperature comprised between 20° C. and 200° C., of an aforesaid liquid or solid mixture comprising an agent capable of structuring and an agent capable of cross-linking on the aforesaid support.

8. Complex obtained by implementing the method according to claim 7.

9. Method for extracting or separating, from an aqueous or organic medium, strategic metals constituted by the rare earths, antimony, indium, beryllium, magnesium, cobalt, niobium, the platinoids, gallium, germanium, tantalum, tungsten, molybdenum, titanium, mercury, cesium, lithium and strontium, said method comprising the step of:
(i) placing an aqueous or organic medium in contact with a complex according to claim 1.

10. Method according to claim 9, also comprising after step (i), a step of recovering strategic metals, in particular rare earths, retained in the aforesaid complex.

11. Phosphorylated calixarene of formula I: 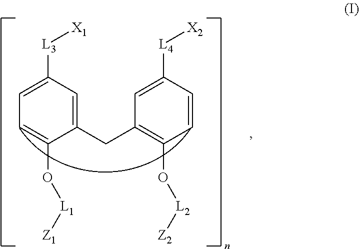
in which:
X₁ and X₂ each represent, independently of one another, H or a 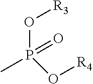
group, in which R₃ and R₄ each represent, independently of one another, H or a (C₁-C₈) alkyl group, provided that X₁ and X₂ do not simultaneously represent H,
L₁, L₂, L₃ et L₄ are spacer groups, selected independently of one another from the group consisting of a (C₃-C₁₀) cycloalkylenyl group, O, NH, —(CH₂)_{q}—, q being an integer from 0 to 12, or from 1 to 12,
Z₁ et Z₂ each represent, independently of one another, a functional group selected from an optionally protected amine group, F, Cl, Br, I, OH, C(═O)H, C(═O)Hal, an aryl group or a substituted aryl group, such as a tosyl group, a diazonium group, an aromatic heterocycle such as a pyrrolyl, furyl, thienyl or pyridinyl group, an optionally protected sulphate or sulphonate group, or a 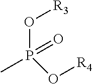
group, in which R₃ et R₄ are as defined above, Z₁ et Z₂ not both being in the 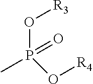
group,
n being an integer from 4 to 100.

12. Phosphorylated calixarene of formula I according to claim 11 for the preparation of a complex whose structuring element is formed by a poly(4-vinylpyridine) and said cross-linking element of said complex is formed by a calix[n]arene in which n is an integer from 4 to 100.
